(19) United States
(12) Patent Application Publication
(10) Pub. No.: US 2024/0408476 A1
(43) Pub. Date: Dec. 12, 2024

(54) HEADWEAR ARTICLES FOR FORCE AND PHYSIOLOGICAL DATA ACQUISITION

(71) Applicant: Iconic X, LLC, Baltimore, MD (US)

(72) Inventors: Steve ARZONI, Lutherville-Timonium, MD (US); Samuel Bo FORREST, Baltimore, MD (US); Christopher Gregg PERRA, Kansas City, MO (US); Rodney Dennis TRAFFORD, Boca Raton, FL (US)

(73) Assignee: Iconic X, LLC, Baltimore, MD (US)

(21) Appl. No.: 18/698,950

(22) PCT Filed: Oct. 6, 2022
(86) PCT No.: PCT/US2022/045955

§ 371 Date: Apr. 5, 2024

Related U.S. Application Data
(60) Provisional application No. 63/253,085, filed on Oct. 6, 2021.
(60) Provisional application No. 63/297,484, filed on Jan. 7, 2022.
(60) Provisional application No. 63/313,110, filed on Feb. 23, 2022.

(51) Int. Cl. A63B 71/10; A63B 24/00

(52) U.S. Cl. CPC A63B 71/10; A63B 24/0062; A63B 2220/40; A63B 2220/53; A63B 2220/833; A63B 2225/20; A63B 2230/50

(57) ABSTRACT

An embodiment of the present disclosure a headwear article for obtaining data from a wearer.

### CROSS-REFERENCE TO RELATED APPLICATIONS

The present application claims the benefit of, and priority to, U.S. Provisional Application No. 63/253,085, filed Oct. 6, 2021, U.S. Provisional Application No. 63/297,484, filed Jan. 7, 2022, and U.S. Provisional Application No. 63/313,110, filed Feb. 23, 2022, the entire contents of each application are incorporated by reference into this application.

### TECHNICAL FIELD

The present disclosure relates to a headwear articles, such as helmets, liner for helmets, and the like, for force and physiological data acquisition.

### BACKGROUND

Safety helmets, in particular, are widely used by athletes in a multitude of sports and construction workers who require protective headgear articles. These helmets often bear the brunt of collisions or other impact forces during use. Further to this, obtaining force impact data during athletic events and construction activities is desirable to determine the durability of the protective headgear article as well as to monitor the health of the device wearer. However, integrating appropriate sensors with a protective headgear article is difficult and not always a predictable endeavor. Moreover, conventional headwear articles often are not always compatible for use with a variety of different safety helmets used in different sports and construction sites. This results in increased costs and limits how effectively force and physiological data can be collected. Durability, reliability, and comfort are important considerations and balancing these factors in a product that meets market needs using available technology is challenging and problematic.

### SUMMARY

An embodiment of the present disclosure is a headwear article configured to be worn under a protective headgear article and/or removably attached to a headwear article. The headwear liner includes a head covering portion for covering a head of wearer and a forward-facing opening for exposing a wearer's face and ears when worn. The headwear liner includes a neck portion coupled to and extending from the head covering portion. The headwear liner also includes an array of force detection sensors disposed along the head covering portion. Each force detection sensor is positioned to obtain force data indicative of forces applied to the each of force detection sensors at different locations of a wearer's head. The headwear liner also includes at least one temperature sensor coupled to the head covering portion at locations that are spaced apart from each of the force detection sensors, the at least one temperature sensor configure to obtain temperature data indicative of the temperature of the wearer of the headwear line. The headwear liner also includes a control unit coupled to the head covering portion proximate to at the neck portion. The control unit is configured for electronic communication with the each one of the force detection sensors and the at least one temperature sensor. The control unit is further configured to store, transmit to a remote device, or both store and transmit to the remote device, the force data and the temperature data.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the present application, there is shown in the drawings, illustrative embodiments of the disclosure. It should be understood, however, that the application is not limited to the precise arrangements and instrumentalities shown. In the drawings:

FIG. 1 is a perspective view of a headwear article according to an embodiment of the present disclosure;

FIG. 2 is a front view of the headwear article shown in FIG. 1;

FIG. 3 is a right-side view of the headwear article shown in FIG. 1;

FIG. 4 is a rear view of the headwear article shown in FIG. 1;

FIG. 5 is a left-side view of the headwear article shown in FIG. 1;

FIG. 6 is a top view of the headwear article shown in FIG. 1;

FIG. 7 is a bottom view of the article shown in FIG. 1;

FIG. 8 is a top rear perspective view of the article shown in FIG. 1; and

FIGS. 9-13 are various views of a sensor array of the headwear article shown in FIG. 1;

FIGS. 9 and 10 are perspective view of a sensor array of the headwear article shown in FIG. 1;

FIG. 11 is a right-side view of the sensor array shown in FIG. 9;

FIG. 12 is a front view of the sensor array shown in FIG. 9;

FIG. 13 is rear view of the sensor array shown in FIG. 9;

FIG. 14 is a schematic showing a system according to an embodiment of the present disclosure;

FIG. 15 is another schematic showing a system according to an embodiment of the present disclosure;

FIG. 16 is a perspective view of a headwear article in accordance with an exemplary embodiment of the present disclosure;

FIG. 17 is a left-side view of the headwear article of FIG. 16;

FIG. 18 is a right-side view of the headwear article of FIG. 16;

FIG. 19 is a rear view of the headwear article of FIG. 16;

FIG. 20 is a top view of the headwear article of FIG. 16;

FIGS. 21 and 22 are perspective view of another embodiment of a sensor array according to an embodiment of the present disclosure;

FIG. 23 is a perspective view of an insert body of the headwear article of FIG. 16;

FIG. 24 is a perspective view of a membrane configured to be injection molded to an insert body of the headwear article of FIG. 1;

FIG. 25 is a perspective view of the headwear article according to another embodiment of the disclosure;

FIG. 26 is a left-side view of the headwear article of FIG. 25;

FIG. 27 is a right-side view of the headwear article of FIG. 25;

FIG. 28 is a rear view of the headwear article of FIG. 25;

FIG. 29 is a top view of the headwear article of FIG. 25;

FIG. 30 is a perspective view of a headwear article in accordance with another embodiment of the disclosure;

FIG. 31 is a left-side view of the headwear article of FIG. 30;

FIG. 32 is a right-side view of the headwear article of FIG. 30;

FIG. 33 is a rear view of the headwear article of FIG. 30; and

FIG. 34 is a top view of the headwear article of FIG. 30.

### DETAILED DESCRIPTION OF ILLUSTRATIVE EMBODIMENTS

Embodiments of the present disclosure include a headwear article, which may be a headwear liner or insert, that is configured to be worn under a protective headgear article and obtain impact force, physiological data, and head position and speed on impact. In one example, the liner that can be used underneath sports helmets. However, the liner can be used with any suitable helmet or other protective headgear.

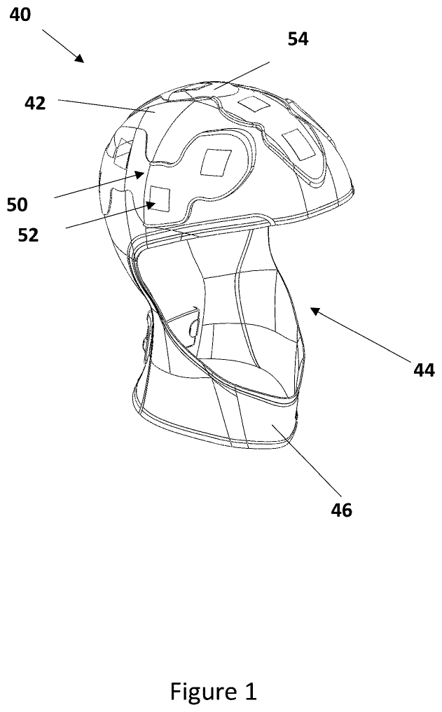

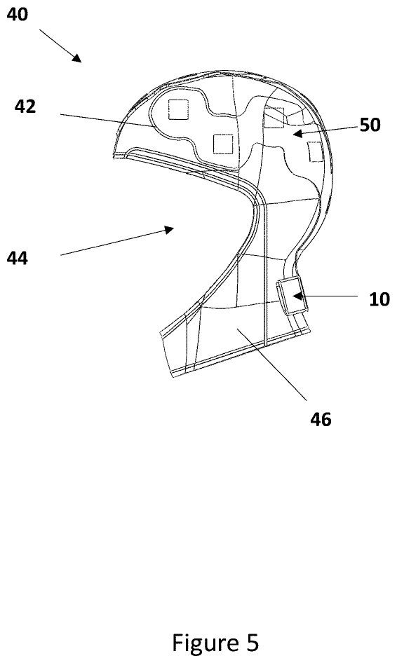

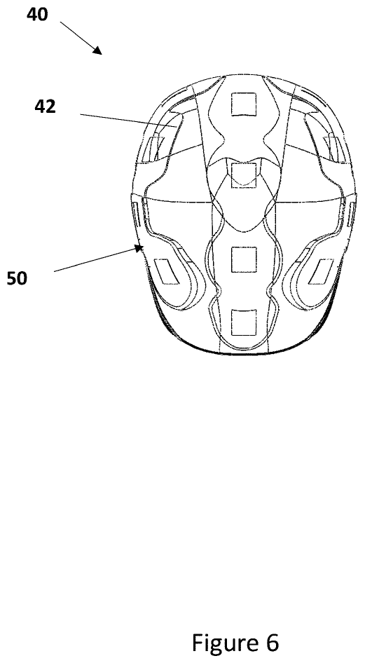

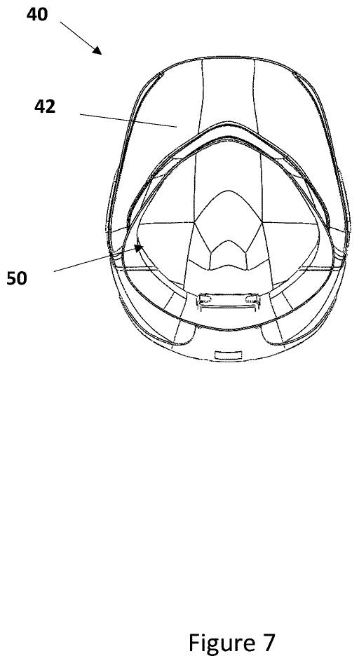

As shown in FIGS. 1-8, an embodiment of the present disclosure is a headwear article 10, in the form of a headwear liner. The headwear liner 40 includes a head covering portion 42 for covering a head of wearer and a forward-facing opening 44 for exposing a wearer's face and ears when worn. The headwear liner 40 includes a neck portion 46 coupled to and extending from the head covering portion 42. The liner 40 may be designed similar to a balaclava where the neck portion encircles the user's neck and the head covering portion 42 covers most of the users head, expect for the face. The head covering portion 42 and the neck portion 46 may be formed from a flexible, breathable textile material. Such a material may be knitted fabric (weft or warp), a woven fabric, or a laminate of one or textile materials.

Referring to FIG. 1-7, the headwear liner 40 also includes a sensor array 50 coupled to the head covering portion. The sensor array 50 includes at least one force detection sensor 52, at least one temperature sensor 54, and a control unit 10. The control unit may include additional sensors, such as accelerometers and gyroscopes, for determining the position and/or movement of the headwear article in three-dimensional space.

The sensor array 50 may include more than one force detection sensor 52. In one example, the sensor array may include between 2 and 15 force detection sensors. Each force detection sensor 52 is positioned to obtain force data indicative of forces applied to the each of force detection sensors at different locations of a wearer's head. The force detection sensors 52 may, in one example, be formed with flexible conductive inks applied to a textile or some other flexible substrate. The flexible conductive inks may be coupled electronically to circuitry in a control unit. The sensor array 50 substrate may differ in construction from the breathable textile material that forms and head covering and neck portion of the headwear liner.

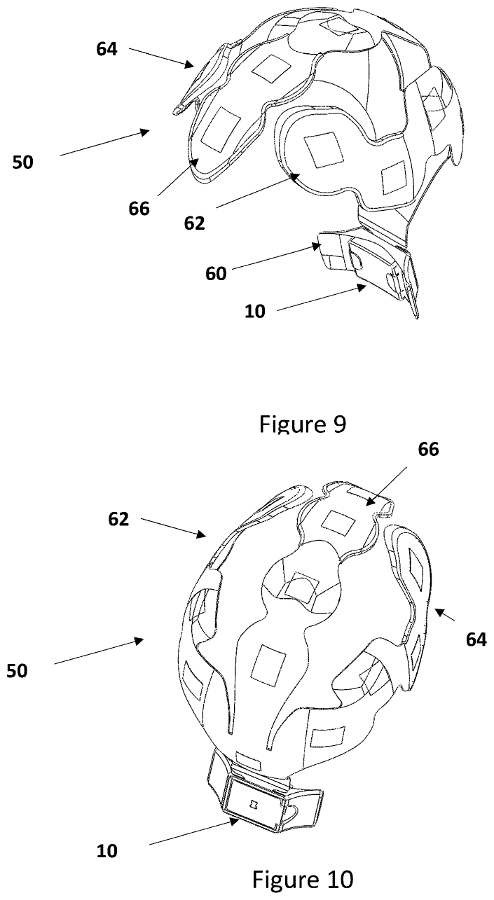

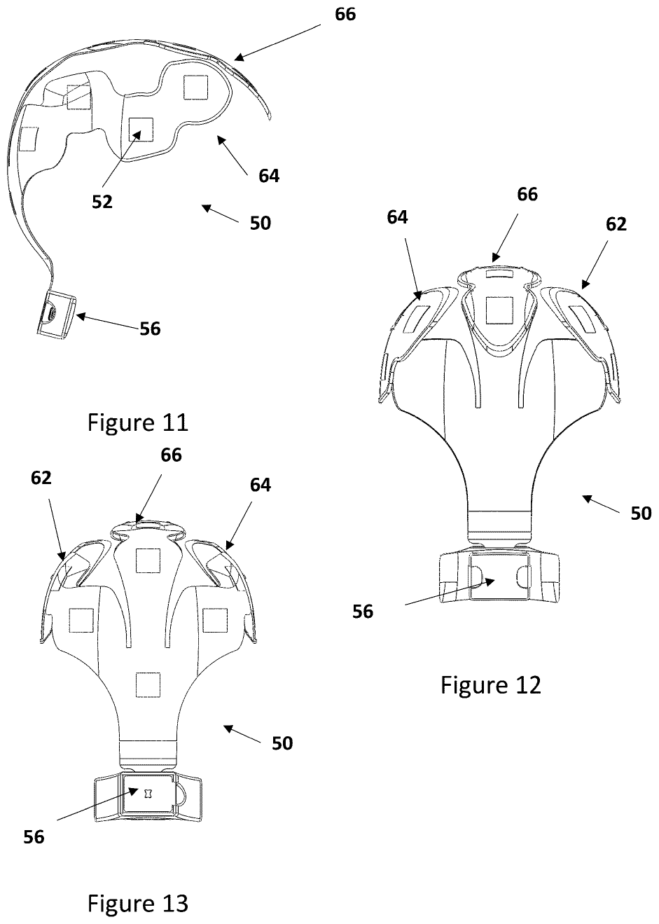

As shown in FIGS. 9-13, the sensor array 50 includes a base 60 located proximate the neck portion, a left side lobe 62 that extends from the base 60 and curves around a left temple portion of the head covering portion, a right side lobe 64 that extends from the base 60 and curves around a right temple portion of the head covering portion, and a crown lobe 66 that extends from the base 60 over a top of the head covering portion and is separate from the left side lobe 62 and the right side lobe 64. Each lobe may include one or more force detection sensors 52. The array substrate and force direction sensors are configured to obtain force data and function reliably after repeated launderings and general use.

Continuing with FIGS. 9-13, the headwear liner 40 may also include a rigidity member (not numbered) coupled to a portion of the sensor array 50 located at a forward area of the head covering portion. The rigidity member is configured to stabilize the location of the sensor arrow on the user head when the headwear liner is worn. The rigidity member may have a tapered edge that extends outward from the array substrate to couple to the head covering portion.

The headwear liner 40 also includes at least one temperature sensor 54 coupled to the head covering portion at locations that are spaced apart from each of the force detection sensors 52. The temperature sensor 54 is configured to obtain temperature data indicative of the temperature of the wearer of the headwear liner. The sensor 54 may be located at proximate the neck. However, the headwear liner 40 can have multiple temperature sensors located around the user's head for additional data collection.

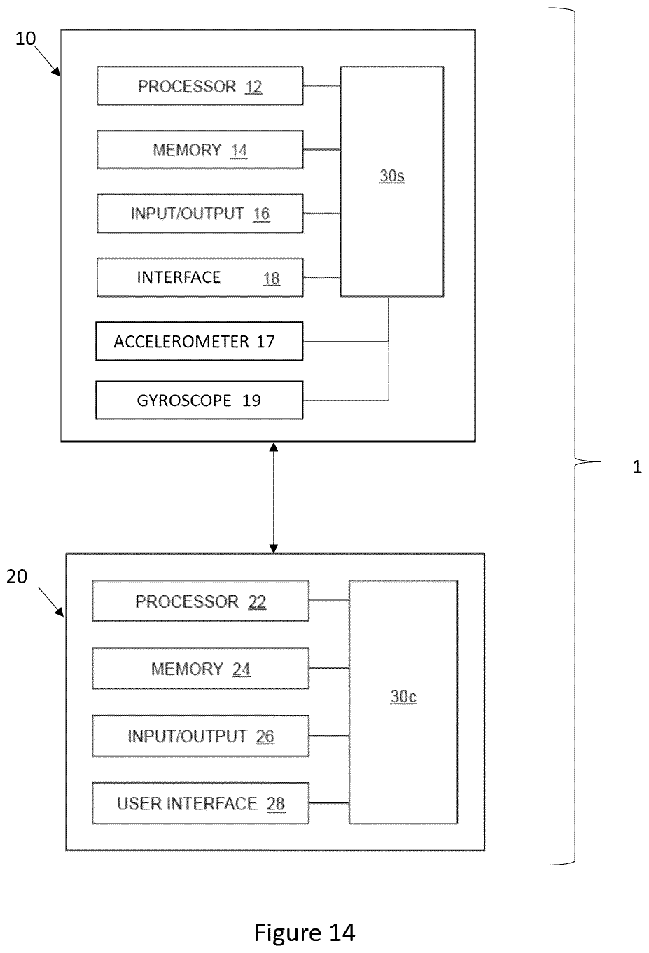

The headwear liner 40 also includes a control unit 10 (schematically shown in FIG. 14). coupled to the head covering portion proximate to at the neck portion. The control unit 10 may include a at least one processor 12, memory 14, a communications unit 16 (e.g. wired and/or wireless), an interface 18, and an optional power source (not shown or numbered). The control unit 10 may include an accelerometer 17 and a gyroscope 19. The accelerometer is configured to obtain data indicative of both head position on impact, and wearer's speed. The power source may be a rechargeable battery. The control unit 10 may include software 30s (firmware or control logic) to facilitate function and operation of the control unit 10 and communication with other sensors in the array. The control unit is configured for electronic communication with the each one of the force detection sensors and the at least one temperature sensor. The control unit is further configured to store, transmit to a remote device, or both store and transmit to the remote device, the force data and the temperature data. The remote device may be a remote smartphone, computer server, or any other computing device that can receive data obtained and stored in the memory of the control unit.

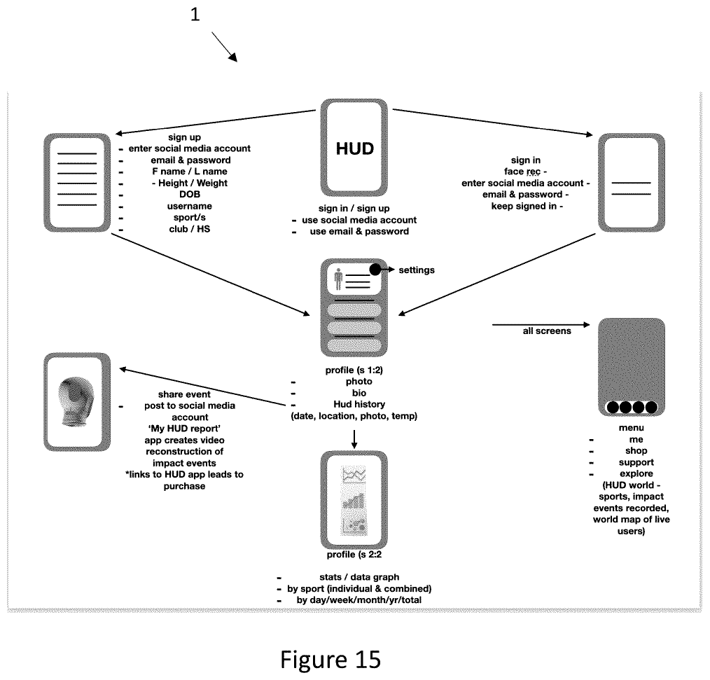

The methods and system described herein related to data flow may be implemented using system 1 as shown in FIGS. 14 and 15. The system 1 include control unit 10, which is coupled to the sensor array as discussed above and a first computing device 20. The controller 10 and first computing device 20 may be linked over various types of communication protocols and networks. An exemplary controller 10 and parent device 20 is shown in FIG. 14. One or more software applications 30s and 30c implemented across control unit 10 and computing device 20. With multiple users, there will be multiple control units in communication with one or more computing devices 20. In addition, the present disclosure describes software applications implemented over system components and configured to execute various steps in the methods described herein. It should be appreciated that a software application can implement steps in the described methods utilizing all of the system components or just portions of the system components. Furthermore, the software applications are described below in singular form. It should be appreciated that multiple software applications may interface to perform the described functions and multiple applications can run on more than one computing device to implement the methodologies described herein.

Continuing with reference to FIG. 14, the system 1 can be implemented via exemplary architecture that includes control unit 10 in electronic communication with one or more computing device 20 via a common communications network, such as, for example, the Internet, Bluetooth protocol, or even via RF communication protocols. Furthermore, one or all of the control units 10 and computing devices 20 can access information on each other. “Access” or “accessing” as used herein can include retrieving information stored in memory on a computing device. For instance, “access” or “accessing” includes sending instructions via the network from control unit 10 to computing device 20 so as to cause information to be transmitted to the memory of the computing device 20 for access locally by the computing device 20. In addition, or alternatively, “access” or “accessing” can include the control unit 10 sending an instruction to computing device 20 to access information stored in the memory of the computing device 20. Reference to control unit 10 and computing device 20 in this paragraph is exemplary and are used to only clarify use of words “access” or accessing.”

In FIG. 14, the first computing device 20 is configured to receive, process, and store various information used to implement one or more software applications, such as client software application 30c. It will be understood that the hardware components of computing device 20 can include any appropriate device, examples of which include a portable computing device, such as a laptop, tablet or smart phone, or other computing devices, such as, a desktop computing device or a server-computing device.

As illustrated in FIG. 14, the first computing device 20 includes one or more processors 22, a memory 24, an input/output 26, and a user interface (UI) 28. It is emphasized that the operation diagram depiction of the computing device 20 is exemplary and not intended to imply a specific implementation and/or configuration. The processor 22, memory 24, input/output portion 26 and user interface 28 can be coupled together to allow communications therebetween and can interface with the client software application 30c. The client software application 30c may include an application programmatic interface (API). As should be appreciated, any of the above components may be distributed across one or more separate devices.

Continuing with FIG. 14, the memory 24 can be volatile (such as some types of RAM), non-volatile (such as ROM, flash memory, etc.), or a combination thereof, depending upon the exact configuration and type of processor 22. The computing device 20 can include additional storage (e.g., removable storage and/or non-removable storage) including, but not limited to, tape, flash memory, smart cards, CD-ROM, digital versatile disks (DVD) or other optical storage, magnetic cassettes, magnetic tape, magnetic storage or other magnetic storage devices, universal serial bus (USB) compatible memory, or any other medium which can be used to store information and which can be accessed by the computing device 20.

Continuing with FIG. 14, in various embodiments, the input/output portion 26 includes an antenna or an electronic connector for wired connection, or a combination thereof. In some implementations, input/output portion 26 can include a receiver and transmitter, transceiver or transmitter-receiver. The input/output portion 26 is capable of receiving and/or providing information pertaining to communication with a network such as, for example, the Internet, or any other communications protocol. As should be appreciated, transmit and receive functionality may also be provided by one or more devices external to the computing device 20. For instance, the input/output portion 26 can be in electronic communication with a receiver.

Referring to FIG. 14, the user interface 28, which can include an input device and/or display (input device and display not shown) that allows a user to communicate with the computing device 20. The user interface 28 can include inputs that provide the ability to control the computing device 20, via, for example, buttons, soft keys, a mouse, voice actuated controls, a touch screen, movement of the computing device 20, visual cues (e.g., moving a hand in front of a camera on the computing device 20), or the like. The user interface 28 can provide outputs, including visual displays. Other outputs can include audio information (e.g., via speaker), mechanically (e.g., via a vibrating mechanism), or a combination thereof. In various configurations, the user interface 28 can include a display, a touch screen, a keyboard, a mouse, a motion detector, a speaker, a microphone, a camera, or any combination thereof. The user interface 28 can further include any suitable device for inputting biometric information, such as, for example, fingerprint information, retinal information, voice information, and/or facial characteristic information, for instance, so as to require specific biometric information for access to the computing device 20. It should be appreciated that the computer devices can operate via any suitable operating system, such as Android, BSD, iOS, Linux, OS X, QNX, Microsoft Windows, Windows Phone, and IBM z/OS. Furthermore, the software application can operate with any of the aforementioned operation systems.

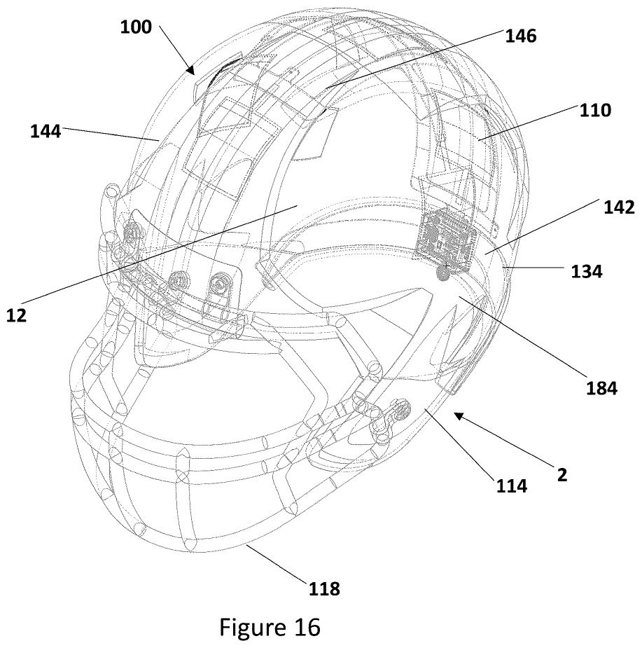

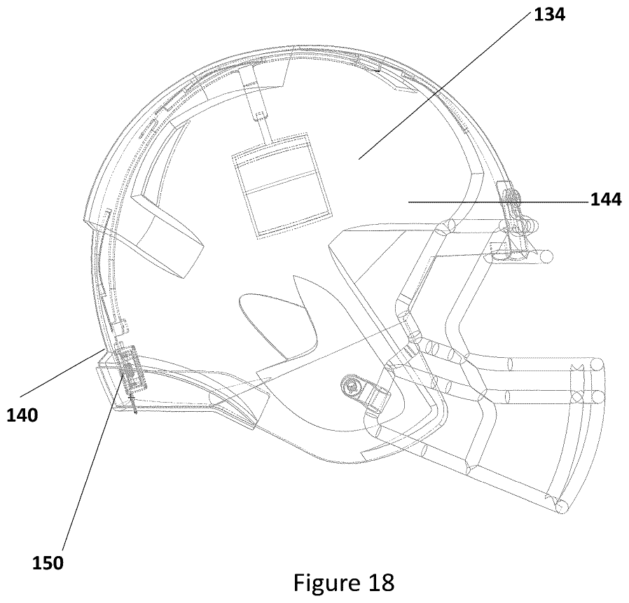

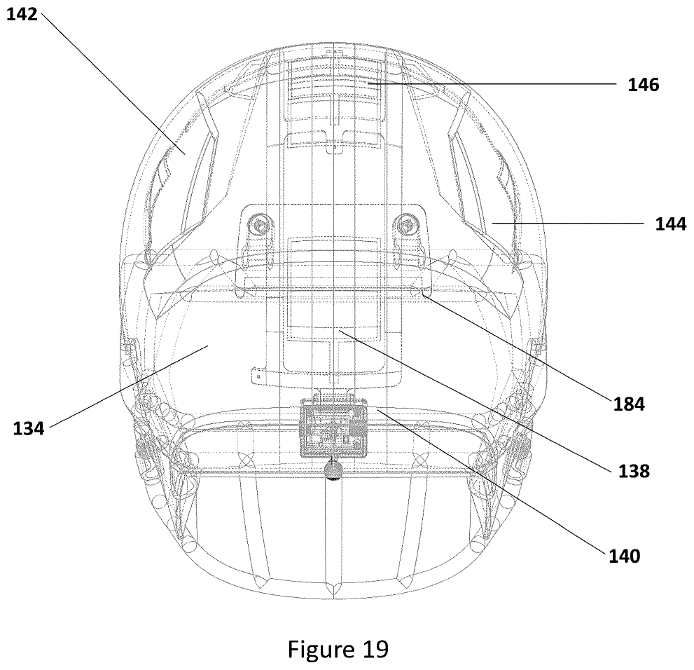

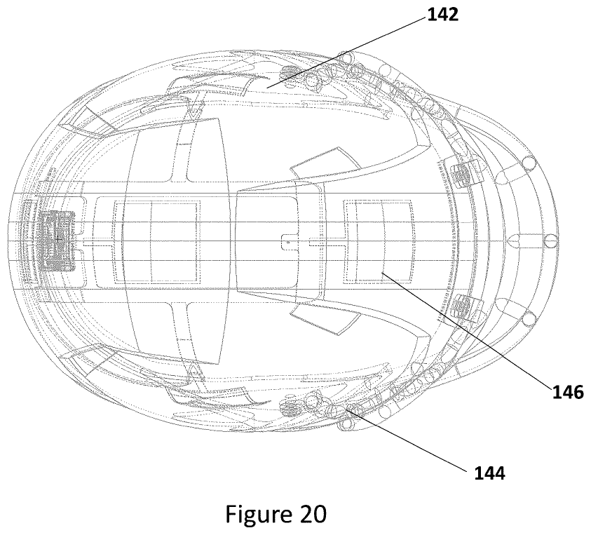

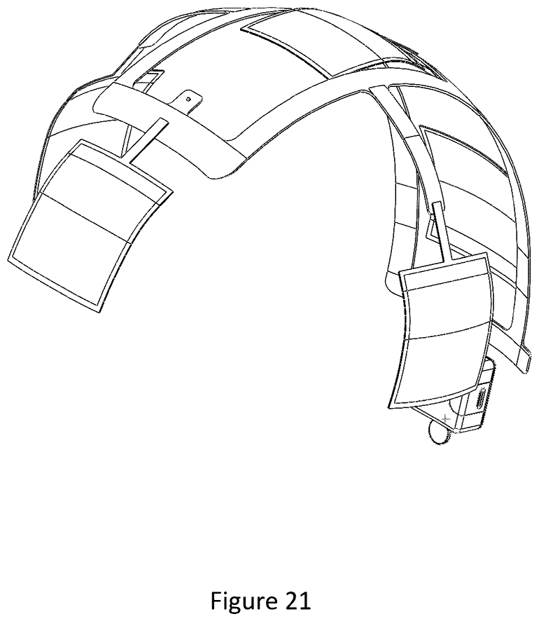

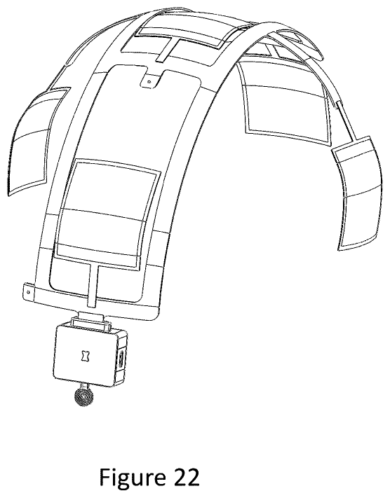

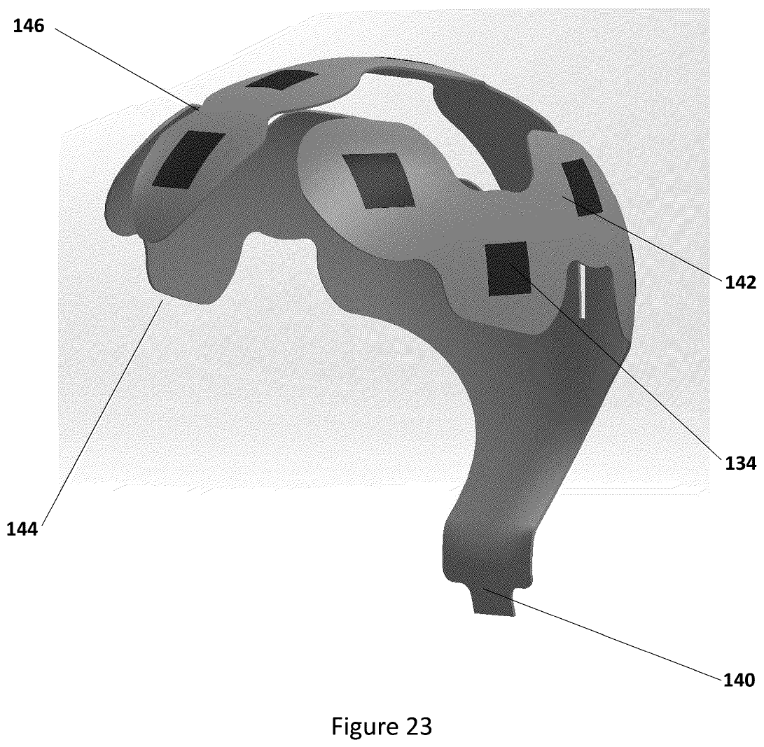

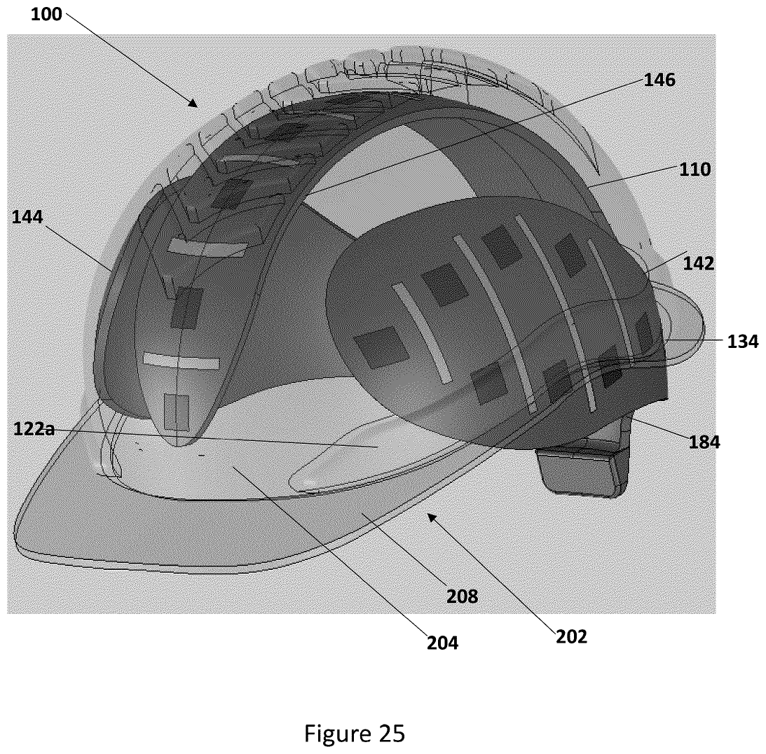

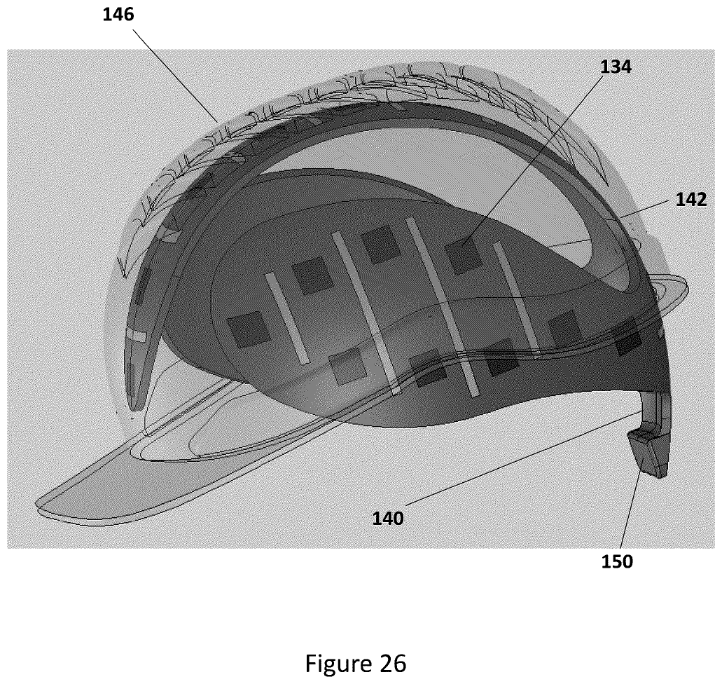

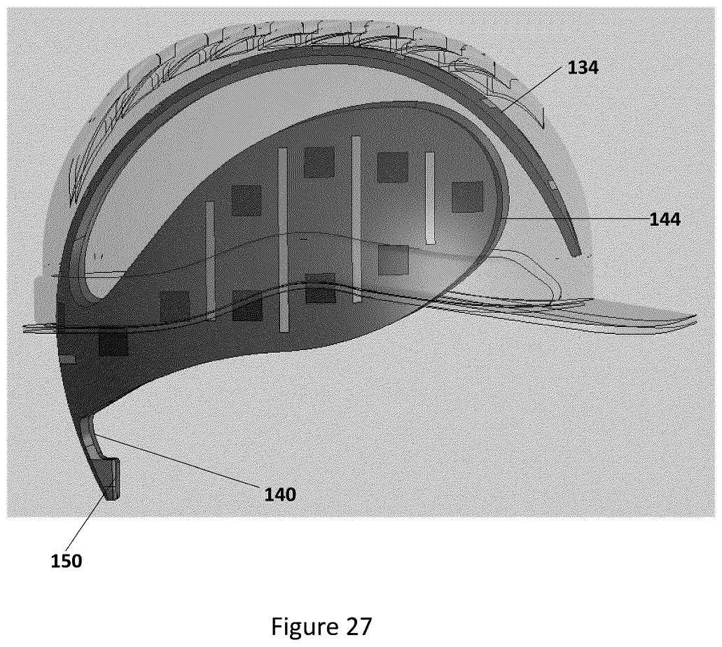

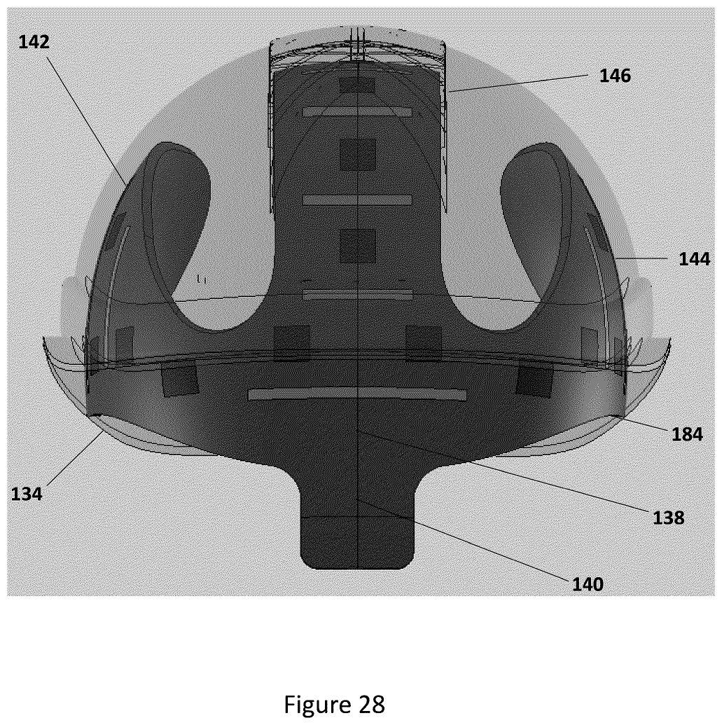

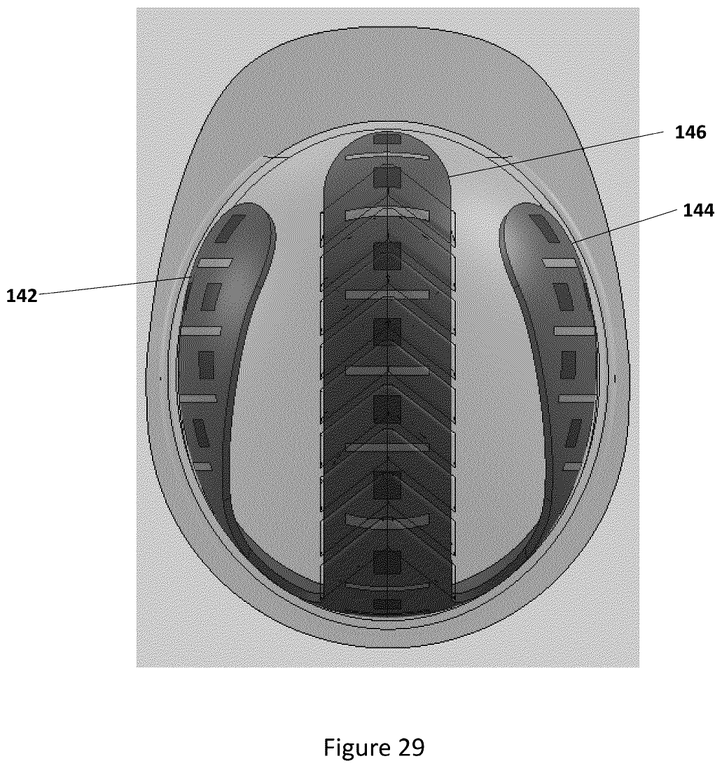

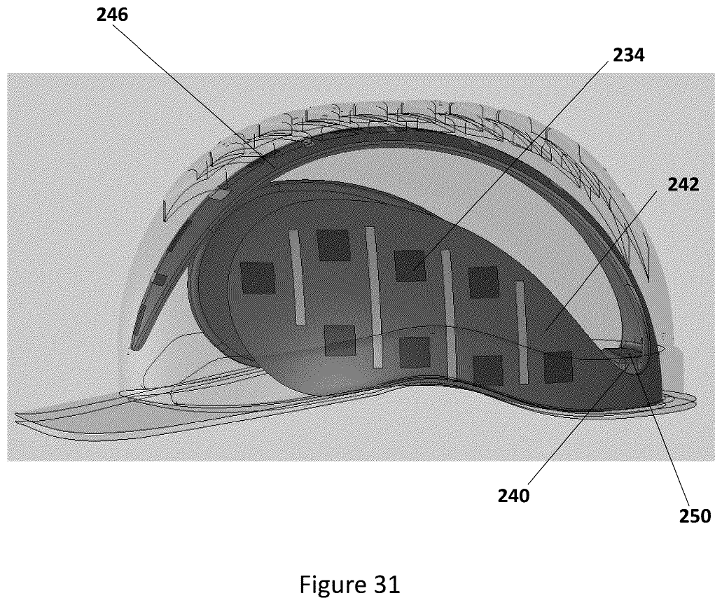

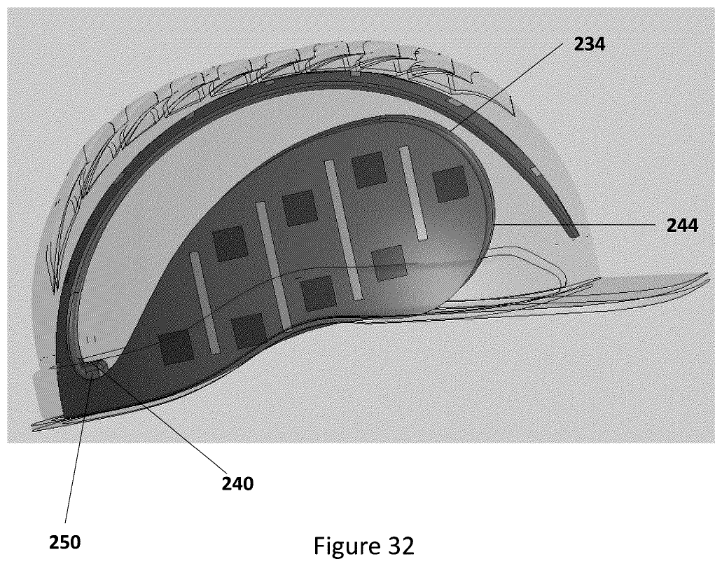

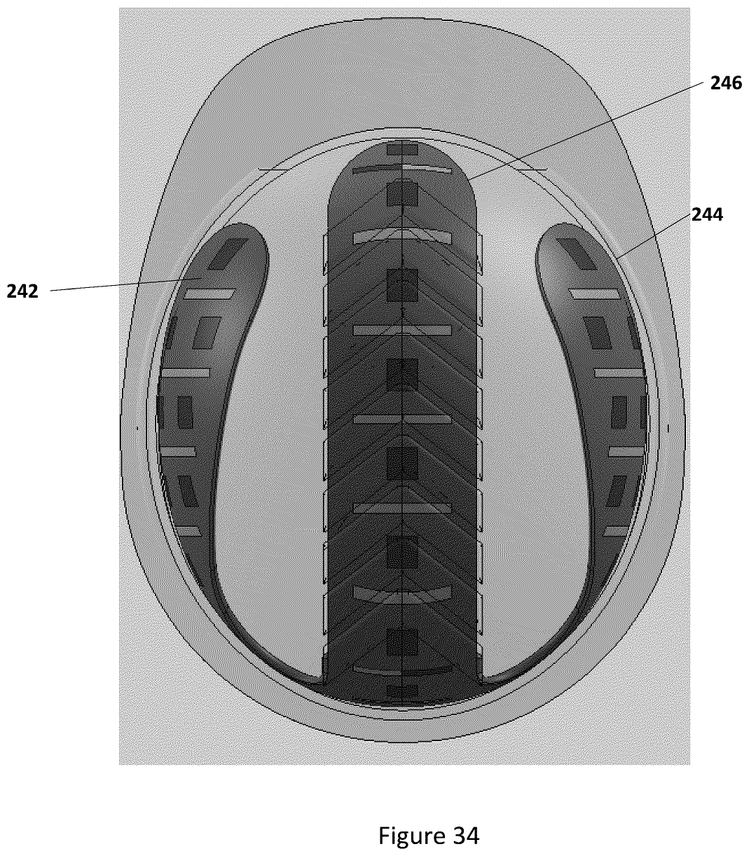

Referring to FIGS. 16-20, there is shown another embodiment of a headwear article 100. FIGS. 16-20 show the headwear article 100, configured as a liner or insert removably attached inside an exemplary protective headgear article 2. The protective headgear article 2 is shown configured as a football helmet. However, it is to be understood that the protective headwear article 2 can also be used with other types protective headgear used by individuals performing various activities including sports, operating motor vehicles, and construction. For example, as shown in FIGS. 25-34, the headwear article 100 can be used with other protective headgear articles such as construction hard hats to facilitate proper work safety protocols on construction sites, oil rigs, and the like. For purposes of clarity, the discussion below of the exemplary embodiment will relate to use of the headwear article with a helmet 2.

As shown in FIGS. 1-5, the helmet 2 includes a shell 114 for protecting the wearer's head and a facemask 118 for protecting the face of the wearer. The helmet 2 further includes a plurality of interior pad members 112 that line the interior of the shell 114. The shell 114 can be structured as any type of helmet shell used in a variety of sports for protecting the head of the wearer. The facemask 118 can be sized and shaped in a variety of different ways based on the needs of a wearer or the activity. For example, the facemask 118 can include a plurality of cross bars, a reflective face shield, or other configurations to further protect the face of the wearer. The plurality of interior pad members 112 can be any type of helmet padding well known in the art. For example, the interior pad members 112 can be configured as foam padding, inflatable padding, webbing, or any other pad suitable for its intended purpose of protecting the wearer's head.

The headwear article 100 includes an insert body 110, at least one force detection sensor 134 positioned along the insert body, at least one temperature sensor 138 positioned along the insert body, and a plurality of attachment members 184 configured to secure the insert body to the protective headgear article 2.

The insert body 110 is configured for removable attachment to an interior of the protective headgear article 2. Accordingly, the insert body 110 has a size and shape to allow for its placement adjacent to the plurality of interior pad members 112 of the protective headgear article 2.

The insert body 110 includes a base 140 located proximate a neck area of the device wearer's head. The insert body 110 further includes a left side lobe 142 that extends from the base 140 and curves adjacent a left temple portion of the protective headgear article 2. Similarly, the insert body 110 includes a right side lobe 144 that extends from the base 140 and curves adjacent a right temple portion of the protective headgear article 2. It is to be understood that the left side lobe 142 and right side lobe 144 are of similar size and shape. The insert body 110 also includes an elongated crown lobe 146 that extends from the base 140 adjacent an upper portion of the protective headgear article 2 and separate from the left side lobe 142 and the right side lobe 144. As further discussed below, the left side lobe 142, right side lobe 144, and elongated crown lobe 146 include one or more force detection sensors 134 for collecting force and/or physiological data.

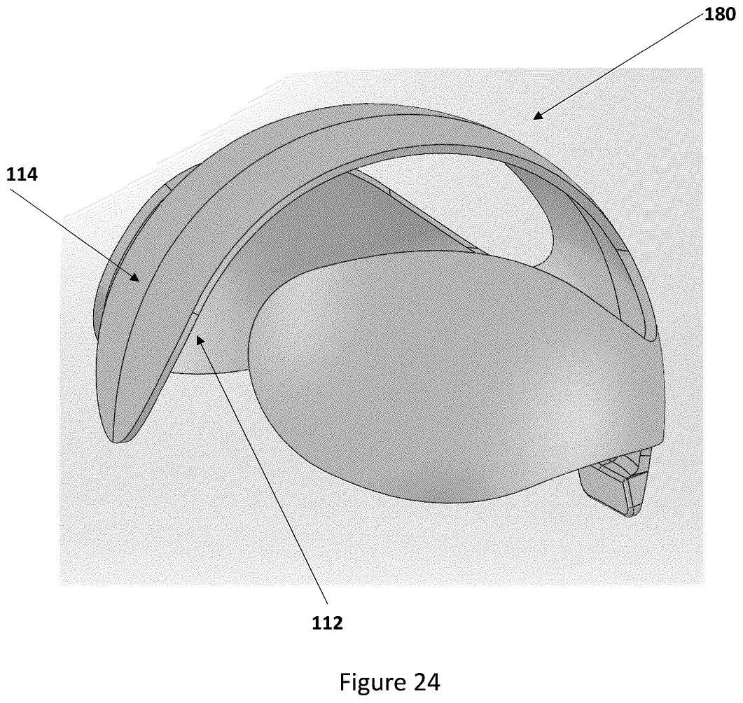

The insert body 110 can be manufactured from a number of materials including polymeric materials such as a silicone membrane, polyisoprene, polyurethane, viscoelastic polymer, polyethylene terephthalate (PET), a polymeric plastic film, and other materials suitable for its intended purpose. In accordance with an aspect, the insert body 110 can also be injection molded with a silicone membrane 180 illustrated in FIG. 24.

The at least one force detection sensor 134 is positioned along the insert body 110. As shown in FIGS. 16-20, the at least one force detection sensor 134 is positioned along the left side lobe 142, right side lobe 144, and elongated crown lobe 146. In accordance with an aspect of the exemplary embodiment, the at least one force detection sensor 134 can be positioned along the insert body 110 as shown. In an alternative configuration as shown in FIG. 24, the at least one force detection sensor 134 can also be positioned along an inner surface 112 of the insert body 110. Alternatively, the at least one force detection sensor 134 can also be positioned along an outer surface 114 of the insert body 110.

In accordance with an aspect, the at least one force detection sensor includes a set of force detection sensors 134 that are configured to obtain force data indicative of forces applied to the headwear article along multiple different directions. For example, the at least one force detection sensor may include between 2 and 28 force detection sensors 134 positioned and spaced throughout the surface of the insert body 110. The force detection sensors 134 may, in one example, be formed with flexible conductive inks applied to a flexible substrate. It is to be understood that the orientation and placement of the at least one force detection sensor 134 can be customized based on user preference and the desired clinical data to be collected. Each of the force detection sensors 134 is configured to obtain force data and function reliably after repeated launderings and general use.

Suitable sensors include the commercially available A502 sensors sold under the trade name FlexiForce®, manufactured and sold by Tekscan. The A502 sensors advantageously have a small size (diameter and thickness) and variable resistance output to applied forces. Additionally, the A502 sensor is square shaped and can easily be integrated into the headwear article without changing the shape or appearance of the headwear article due to its flexibility. However, it is to be understood that any suitable flexible sensor generally known in the art for measuring force and/or physiological data can be used with the headwear article 100.

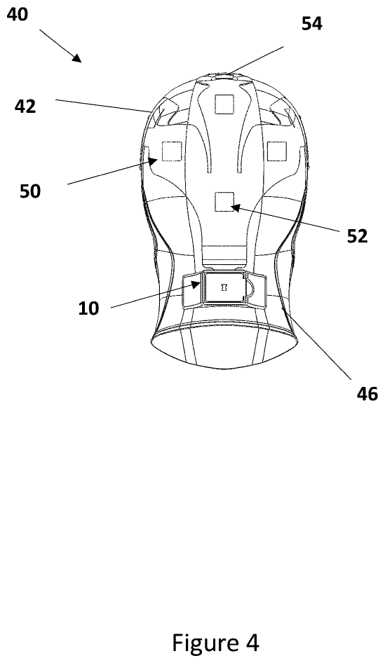

As discussed above, the at least one temperature sensor 138 is positioned along the insert body 110. For example, as shown in FIG. 4, the temperature sensor 138 is positioned proximate a neck area of the device wearer's head. Additionally, the temperature sensor 138 is spaced apart from each of the force detection sensors 134. In general, the temperature sensor 138 is configured to obtain temperature data indicative of the body temperature of the wearer of the headwear article 100. The temperature sensor 138 may, in one example, be formed with flexible conductive inks applied to a flexible substrate. Similar to the force detection sensors 134, the orientation and placement of the temperature sensor 138 can be customized based on user preference and the desired clinical data to be collected. In accordance with an aspect, the headwear article headwear liner 100 can include multiple temperature sensors 138 positioned along various locations of the insert body 110 to collect additional temperature data. Each of the temperature sensors 138 is configured to obtain temperature data and function reliably after repeated launderings and general use.

In general, the force detection sensor 134 and the temperature sensor (or sensors) 138 may be selectively positioned into a plurality of locations to collect force and temperature data at a plurality of locations in a serial manner (e.g., one at a time, etc.) or a plurality of locations simultaneously. The sensors can be positioned to collect data around specific predetermined areas of the protective headgear article 2. For example, data collection may focus only on specific areas of the protective headgear based on the desired area to be analyzed.

The headwear article 100 includes a plurality of attachment members 184 configured to secure the insert body 110 to the protective headgear article 2. As shown in FIGS. 16-20, each of the plurality of attachment members 184 is disposed between a pair of force detection sensors 134. However, it is to be understood that the orientation and position of each of the plurality of attachment members 184 can be customized based on user preference and the desired application of the headwear article 100. For example, using the headwear article with certain types of protective headgear articles 10 may require additional attachment members 184 to ensure that the insert body 110 is secured to the protective headgear article 2 after repeated use under varying conditions.

In accordance with an embodiment, the attachment member 184 is an adhesive gripping member. However, the attachment member 184 can be any type of fastener or gripping member that can perform the function of securing the insert body 110 to the protective headgear article 2. For example, the attachment member 184 can utilize nano-grip technology to secure the insert body 110 to the protective headgear article 2 without leaving any marks on the inner surface of the protective headgear article.

In accordance with another embodiment, the attachment member 184 is a fastener for mechanically coupling the insert body 110 to the protective headgear article 2. For example, the fasteners can be adjustably positioned or oriented to facilitate mechanical coupling of the headwear article to specific types of protective headgear articles available in the industry, including but not limited to, those provided by specific manufacturers. That is, the headwear article 100 can be custom-fit to specific types of protective headgear articles. It is to be understood that the attachment member can be any type of fastener that can perform the function of mechanically coupling the insert body 110 to the protective headgear article 2. The fastener can include, for example, a clasp, clamp, buckle, clip, hook, tab, or a component of such or a similar device.

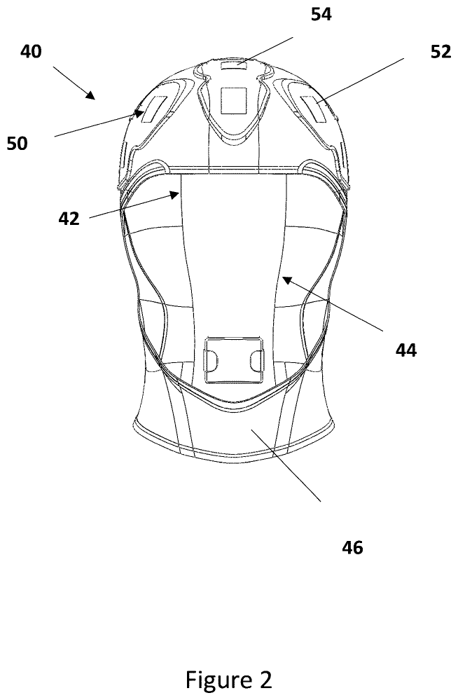

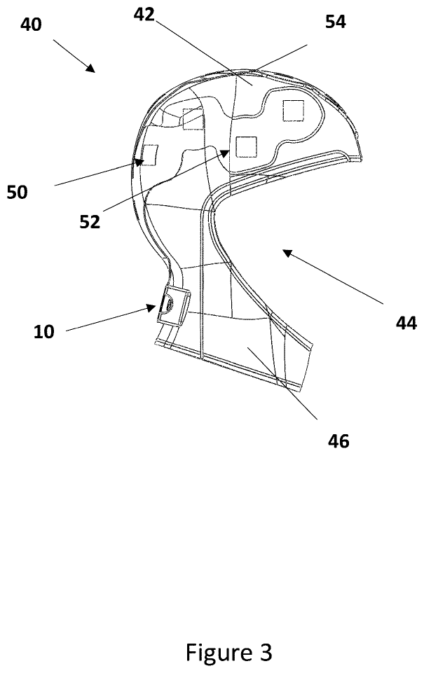

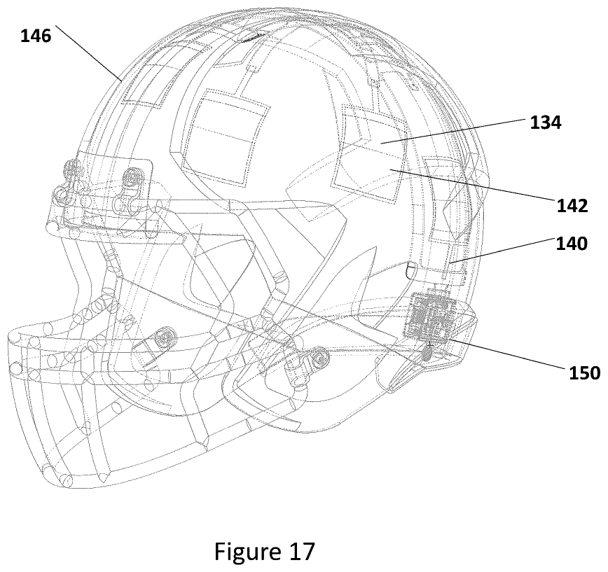

Referring now to FIGS. 2 and 3, the headwear article 100 further includes a circuit board 150 operatively connected with the at least one force detection sensor 134 and the at least one temperature sensor 138. The circuit board 150 is configured to store, transmit to a remote device, or both store and transmit to the remote device, the force data and the temperature data collected from the at least one force detection sensor 134 and the at least one temperature sensor 138. In accordance with an aspect, the circuit board 150 is configured to remotely transmit the force data and the temperature data in real time. Alternatively, the circuit board 150 can collect and store the force data and temperature data in real time, thereby allowing the collected data to be transmitted and analyzed later. As shown in FIGS. 16 and 17, the circuit board 150 can be removably coupled to the base 140 of the insert body 110. However, it is to be understood that the circuit board 150 can be adjustably positioned based on a desired orientation so long as the circuit board is operatively connected to the force detection sensor 134 and the temperature sensor 138 for accurate data collection.

The headwear article 100 further includes a power source (not shown) removably coupled to the circuit board 150. For example, the power source can be a rechargeable battery. In accordance with another aspect, the circuit board 150 further includes a multi-color LED, Bluetooth antenna for communication and transmission of collected data, and storage for collecting force data and temperature data.

The headwear articleheadwear100 described above is attached and/or retrofit to a conventional headgear article 2. The at least one force detection sensor 134 and the at least one temperature sensor 138 can be positioned in a predetermined orientation along an inner or outer surface of the insert body 110 based on the desired data to be collected. The at least one force detection sensor 134 and the at least one temperature sensor 138 are then soldered to the circuit board 150. Thereafter, a power source can be soldered to the circuit board 150. A silicone membrane 180 (FIG. 24) can then be injection molded onto the circuit board 150 and the sensors positioned on the insert body 110. The plurality of attachment members 184 are then bonded to the silicone membrane to finalize assembly of the headwear article 100. The fully assembled headwear article 100 is then attached to the protective headgear article 2 via the plurality of attachment members 184. It is to be understood that the process for assembling the headwear article as well as the process for attaching the assembled headwear article to the protective headgear article 2 can be repeated by a user until an optimal configuration is achieved.

The components of the headwear article are hand washable and waterproof. The components of the headwear article are also preferably resistant to adverse conditions including, but not limited to, variable temperatures, adverse weather, impact forces, and degradation over time.

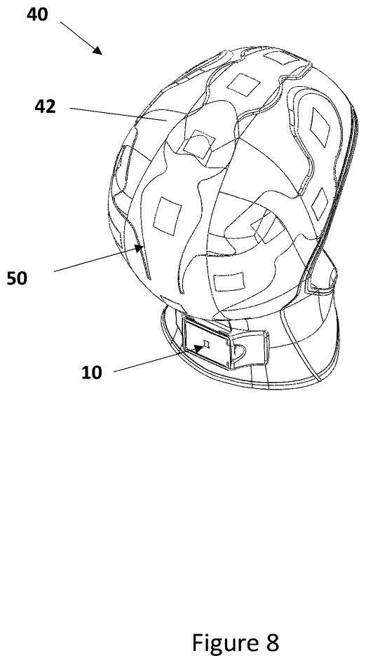

The headwear article 100 can further include a control unit 170 (schematically shown in FIG. 8) that is remotely connected to the circuit board 150 for receiving collected and/or stored force data and temperature data. The control unit 170 may include at least one processor 171, memory 172, a communications unit 173 (e.g., wired or wireless), an interface 174, and an optional power source (not shown). The control unit 170 is remotely connected to an accelerometer 175 and a gyroscope 176 positioned on the circuit board 150. The accelerometer 175 is configured to obtain data indicative of both head position on impact, and wearer's speed. In accordance with another aspect, the control unit 170 may include software 177 (firmware or control logic) to facilitate function and operation of the control unit 170 and communication with the circuit board 150 and sensors 134, 138 operatively connected to the circuit board 150. The control unit 170 may be located on a remote smartphone, computer server, or any other computing device that can receive data obtained and stored in the memory of the circuit board and/or the control unit 170.

The methods and system described herein related to data flow may be implemented using system 1 similar to that shown in FIGS. 14 and 15. The system 1 includes the control unit which is remotely connected to the circuit board as discussed above and thereby operatively connected to sensors positioned on the insert body 110. The system 1 also includes a first computing device in communication with the control unit. Specifically, the control unit and first computing device may be linked over various types of communication protocols and networks. An exemplary control unit and first computing device is shown in FIGS. 14 and 15. In accordance with an aspect, one or more software applications and can be implemented across control unit and first computing device. It is further contemplated that with multiple users, multiple control units will be in communication with one or more computing devices.

Referring now to FIGS. 25-29, the headwear article 100 discussed above is shown in use with another exemplary protective headgear article. Namely, FIGS. 25-29show the headwear article headwear liner 100 attached to a protective headgear article, i.e., hard hat 202. The hard hat 202 includes a shell 204 for protecting the wearer's head and a peak 208 for protecting the eyes and face of the wearer. The hard hat 202 further can include a plurality of interior pads 122a that line the interior of the shell 204. The shell 204 can be sized and shaped in a variety of different ways based on the needs of a wearer or the type of construction activity. For example, the hard hat 200 can include an adjustable chin strap, ventilation openings, and be manufactured from a variety of different materials that provide voltage protection. The plurality of interior pad members 122a can be any type of padding well known in the art. For example, the plurality of interior pad members 122a can be configured as foam padding, inflatable padding, webbing, or any other pad suitable for its intended purpose of protecting the wearer's head.

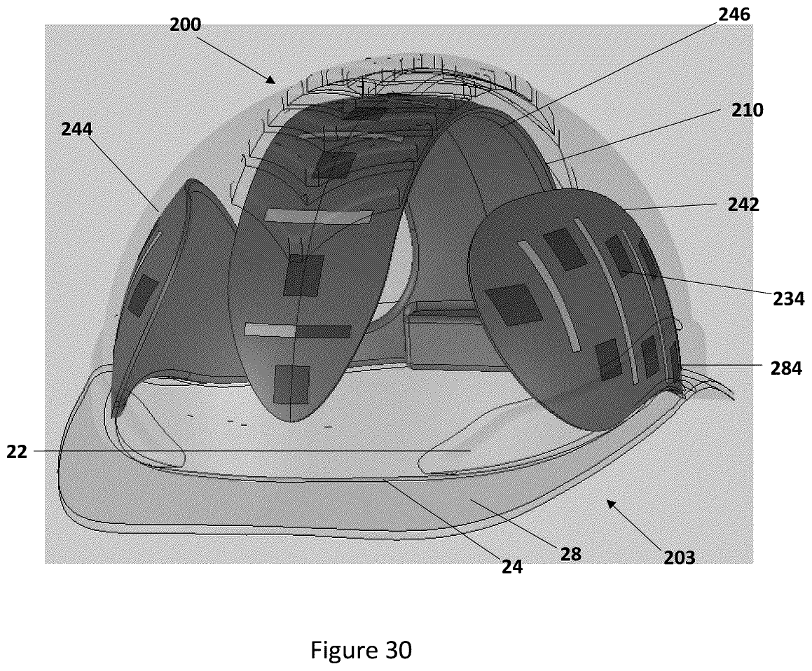

Referring now to FIGS. 30-34, there is shown a headwear article 200 in accordance with another exemplary embodiment of the present disclosure. As shown in FIGS. 30-34, the headwear article 200 is attached to the protective headgear article, i.e., hard hat 203 discussed above. The headwear article 200 is similar to headwear article 200 except as specifically described herein.

The headwear article 200 includes an insert body 210, at least one force detection sensor 234 positioned along the insert body, at least one temperature sensor 238 positioned along the insert body, and a plurality of attachment members 284 configured to secure the insert body to the protective headgear article 20.

The insert body 210 includes a base 240 located proximate a neck area of the device wearer's head. The insert body 210 further includes a left side lobe 242 that extends from the base 240 and curves adjacent a left temple portion of the protective headgear article 20. Similarly, the insert body 210 includes a right side lobe 244 that extends from the base 240 and curves adjacent a right temple portion of the protective headgear article 203. The insert body 210 also includes an elongated crown lobe 246 that extends from the base 240 adjacent an upper portion of the protective headgear article 203 and separate from the left side lobe 242 and the right side lobe 244.

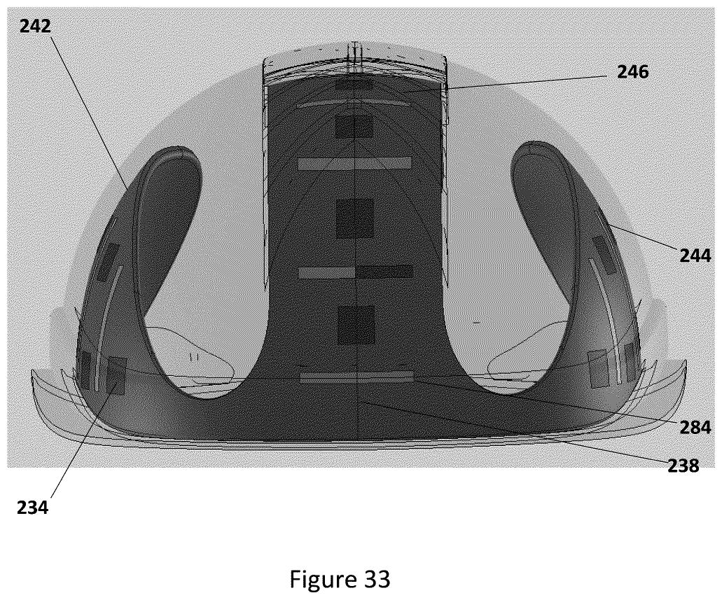

As shown in FIGS. 30-34, the at least one force detection sensor 234 is positioned along the left side lobe 242, right side lobe 244, and elongated crown lobe 246. As shown in FIG. 33, the at least one temperature sensor 238 is positioned along the insert body 210.

Referring now to FIGS. 30-32, the headwear article 200 further includes a circuit board 250 operatively connected with the at least one force detection sensor 234 and the at least one temperature sensor 238. The circuit board 250 can be removably coupled to the base 240 of the insert body 210. It is to be further appreciated that headwear article 200 can function similarly to headwear article 100 as previously discussed.

Embodiments of the present disclosure include a system and method for obtaining and processing data obtained by the headwear article during use. As shown in figures and described above, as the headwear article is being worn and impacts experienced by the wearer, data obtained from the various sensors are stored in the memory of the controller. The data may include forces applied, temperature of the wearer, directional data of wearer, and speed data wearer. In use or after an event, the data stored in the memory unit can be transmitted via a communications protocol (e.g. via Bluetooth, LAN, WAN, RF) to a memory unit 24 of a first computing device 20. For example, the headwear article can transmit data to parent device 20 when they are within a certain range of each other. The parent device 20 can be then transmit the data to a backend server for storage and use. The first computing device 20 includes a software application 30s, when executed by a processor 22 therein, manages, processes and displays the obtained data for the user.

In an embodiment of the present disclosure, as the headwear is being worn and impact forces are experienced by the wearer, data obtained from the various sensors are stored in the memory of the circuit board and/or control unit. The data may be collected in real time and can include data on forces applied, temperature of the wearer, directional data of the wearer, and speed data of the wearer. Whether the headwear article is in use or after operation, the collected and/or stored data can be transmitted via a communications protocol (e.g., via Bluetooth, LAN, WAN, RF) to a memory of a first computing device. For example, the headwear article can transmit data to the first computing device when within a certain distance of one another. The first computing device can subsequently transmit the data again to a backend server for further storage and analysis. The first computing device includes a software application, when executed by a processor therein, manages, processes and displays the obtained data for the user.

In the context of a sporting event, a group of multiple wearers may adorn the headwear article or multiple headwear articles. In such an exemplary embodiment, the force and temperature data can be collected in real time by the sensors. The collected data can be transmitted via communications protocol, to a first computing device located along a sideline of a playing field for further analysis. For example, the first computing device can store, process, and visualize the data for observation and use by coaching staff, medical personnel and others.

For example, in accordance with an aspect, the headwear article is configured to measure impact forces and accelerations to the head of a wearer in cases of impacts or collisions to the head such as collisions between players during a sporting activity. The sensors are configured to measure impact data regarding an impact force applied to the protective headgear article. When a control unit in electronic communication with the circuit board and sensors measures forces beyond a pre-determined threshold, a visual or auditory indication can be provided to alert coaching staff, medical personnel or others to check for damages and/or the wearer for injuries such as a concussion, neck injury, and the like. The transmission of the visual or auditory indication can be customized to notify specific personnel based on a scheduled event or sporting event.

In another embodiment, in the event context, a team of wearers will adorn the headwear article. The data will be obtained by the sensors. Then, that data will be transmitted via communications protocol, to a parent device located along the sideline of the playing field. The parent device can store, process, and visualize the data for observation and use by the coaching staff and others.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1. A headwear article configured to be worn under an outer protective headgear article, comprising:
a head covering portion for covering a head of wearer, the head covering portion having a forward-facing opening for exposing a wearer's face and ears when worn;
a neck portion coupled to and extending from the head covering portion;
a sensor array located along the head covering portion, the sensor array having a set of force detection sensors, wherein the set of force detection sensors are positioned on the sensor array in order to obtain force data indicative of forces applied to the headwear article along multiple different directions;
at least one temperature sensor coupled to the head covering portion at a location that is spaced apart from each of the force detection sensors, the at least one temperature sensor configured to obtain temperature data indicative of the body temperature of the wearer of the headwear liner; and
a control unit configured for electronic communication with the each one of the force detection sensors and the at least one temperature sensor, the control unit further configured to store, transmit to a remote device, or both store and transmit to the remote device, the force data and the temperature data.

2. The headwear article according to claim 1, further comprising a rigidity member coupled to a portion of the sensor array located at a forward area of the head covering portion, wherein the rigidity member is configured to stabilize the location of the sensor array on the user's head when the headwear article is worn.

3. The headwear article according to claim 1 or claim 2, wherein the sensor array includes a base, a left side lobe that extends from the base and curves around a left temple portion of the head covering portion, a right side lobe that extends from the base and curves around a right temple portion of the head covering portion, and a crown lobe that extends from the base over a top of the head covering portion and is separate from the left side lobe and the right side lobe, wherein the left side lobe, the crown lobe, and the right side lobe each include one or more force detection sensors.

4. The headwear article of any one of the claims 1 to 3, wherein each force direction sensor is configured to obtain force data after repeated launderings.

5. The headwear article of any one of the claims 1 to 5, wherein the at least one temperature sensor is configured to obtain temperature data after repeated launderings.

6. The headwear article of any one of the claims 1 to 5, wherein the at least one temperature sensor and the force direction sensor are flexible.

7. The headwear article of any one of the claims 1 to 6, wherein the head covering portion and the neck portion are formed from a breathable textile material.

8. The headwear article of claim 7, wherein the sensor array includes a substrate that differs in construction and content from the breathable textile material.

9. The headwear article of any one of the claims 1 to 8, further comprising at least one accelerometer.

10. The headwear article of any one of the claims 1 to 9, further comprising at least one gyroscope.

11. The headwear article of any one of the claims 1 to 10 formed as a headwear liner.

12. A method of obtaining data from a wearer of the headwear article according to any one of the claims 1 to 11.

13. A system and method as shown in Figures and described in the specification and appendix.

14. A system, comprising:
a headwear article according to any one the claims 1 to 11; and
a first computing device including a processor, a memory, and a communication unit, wherein the communication unit obtains data from the control unit of the headwear article, and the processor is configured to process and display the obtained data via a user interface.

15. A headwear article liner configured to be attached to a protective headgear article having a plurality of interior pad members each configured to contact a wearer's head, the headwear article liner comprising:
an insert body configured for removable attachment to an interior of the protective headgear article, the insert body having a size and shape selected to permit its placement adjacent to the plurality of interior pad members;
at least one force detection sensor positioned along the insert body, the at least one force detection sensor configured to obtain force data indicative of forces applied to the headwear article liner along multiple different directions;
at least one temperature sensor positioned along the insert body, the at least one temperature sensor configured to obtain temperature data indicative of the body temperature of the wearer of the headwear article liner; and
a plurality of attachment members configured to secure the insert body to the protective headgear article.

16. The headwear article liner according to claim 15, further comprising a circuit board operatively connected with the at least one force detection sensor and the at least one temperature sensor, the circuit board further configured to store, transmit to a remote device, or both store and transmit to the remote device, the force data and the temperature data.

17. The headwear article liner according to claims 15 to 16, wherein the insert body includes:
a base;
a left side lobe that extends from the base and curves adjacent a left temple portion of the protective headgear article;
a right side lobe that extends from the base and curves adjacent a right temple portion of the protective headgear article; and
an elongated crown lobe that extends from the base adjacent a top of the protective headgear article and separate from the left side lobe and the right side lobe, and
wherein the left side lobe, the right side lobe, and the elongated crown lobe include one or more force detection sensors.

18. The headwear article liner according to claim 15, wherein the at least one force detection sensor is positioned along an inner surface of the insert body.

19. The headwear article liner according to claim 15, wherein the at least one force detection sensor is positioned along an outer surface of the insert body.

20. The headwear article liner according to claim 17, wherein the circuit board is coupled to the base of the insert body.

21. The headwear article liner according to claim 16, wherein the circuit board is configured to remotely transmit the force data and the temperature data in real time.

22. The headwear article liner according to claim 16, wherein the circuit board is configured to collect and store the force data and the temperature data.

23. The headwear article liner according to claim 16, further comprising a power source removably coupled to the circuit board.

24. The headwear article liner of any one of the claims 15 to 23, wherein the at least one force detection sensor is configured to obtain force data after repeated launderings.

25. The headwear article liner of any one of the claims 15 to 240, wherein the at least one temperature sensor is configured to obtain temperature data after repeated launderings.

26. The headwear article liner of any one of the claims 15 to 25, wherein the at least one temperature sensor and the at least one force detection sensor are flexible.

27. The headwear article liner according to claim 15, wherein each of the plurality of attachment members is an adhesive gripping member.

28. The headwear article liner according to claim 15, wherein each of the plurality of attachment members is a fastener for mechanically coupling the insert body to the protective headgear article.

29. The headwear article liner according to claim 15, wherein each of the plurality of attachment members is disposed between a pair of force detection sensors.

30. The headwear article liner according to claim 15, wherein the insert body is formed from a silicone material.

31. The headwear article liner of any one of the claims 15 to 30, further comprising at least one accelerometer.

32. The headwear article liner of any one of the claims 15 to 31, further comprising at least one gyroscope.

33. A method of obtaining data from a wearer of the headwear article liner according to any one of the claims 15 to 32.

34. A system and method as shown in Figures and described in the specification and appendix.

35. A system, comprising:
a headwear article liner according to any one of the claims 15 to 33; and
a first computing device including a processor, a memory, and a communication unit, wherein the communication unit obtains data from a control unit of the headwear article, and the processor is configured to process and display the obtained data via a user interface.
